(19) United States
(12) Patent Application Publication
(10) Pub. No.: US 2026/0186985 A1
(43) Pub. Date: Jul. 2, 2026

(54) INTERCONNECTION PLATFORM FOR INTERNAL COMMUNICATIONS AMONG MULTIPLE APPLICATIONS OF PHYSIOLOGICAL MONITOR

(71) Applicant: BROADSIMS INC., Zhubie City (TW)

(72) Inventors: JUI-YUAN YU, Hsinchu City (TW); PENG-HSIANG WANG, Zhubei City (TW)

(21) Appl. No.: 19/431,193

(22) Filed: Dec. 23, 2025

(30) Foreign Application Priority Data
Dec. 31, 2024 (TW) 113151711

(51) Int. Cl. G06F 13/14; A61B 5/00

(52) U.S. Cl. CPC G06F 13/14; A61B 5/002; G06F 2213/40

(57) ABSTRACT

The present invention is an interconnection platform for internal communications among multiple applications of a physiological monitor, comprising a physiological information application device. The physiological information application device performs a connection service through a server application and a binding service through a client application, enabling interconnection between the server application and the client application. This facilitates data exchange and application between the client application and server application. Consequently, the physiological information application device avoids transferring data to external systems, thereby reducing the risk of data omissions and theft, and enhancing data security.

### CROSS REFERENCE TO RELATED APPLICATION(S)

This application claims the priority to patent application No. 113151711 filed in Taiwan on Nov. 27, 2025, which is hereby incorporated in its entirety by reference into the present application.

### BACKGROUND OF THE INVENTION

### 1. Field of the Invention

The present invention relates to an application platform, particularly to an interconnection platform for internal communications among multiple applications of a physiological monitor.

### 2. Description of the Related Art

With the advancement of computer science and technology, medical measurement data and manual assessment data have achieved rapid development in subsequent data re-application. Driven by hospitals' pursuit of paperless operations and continuous workflow optimization, the demand for collecting and uploading data to hospital information systems has become widespread. However, since each hospital deploys its own information system and communication protocols, challenges and obstacles arise during data integration and interoperability. Simultaneously, the burgeoning development of Artificial Intelligence (AI) and its continuously enhanced reasoning capabilities have spurred the emergence of AI-assisted diagnostic and early warning software. Similarly, these applications also encounter challenges and obstacles in data integration and interoperability.

Generally, after medical devices collect signal data, numerical data, audio data, or image data, subsequent applications require transferring the collected data to an external information device and corresponding software to complete the process. For example, collected data is transmitted to the hospital's information system. The hospital system then must transfer the data to an external information device to interface with corresponding auxiliary diagnostic analysis software or early warning software for completing nursing assessment forms, such as those for coma scale, limb muscle strength, pupil size, and other nursing evaluations. Because the hospital's information system must also transfer the data to an external information device and corresponding software to complete the process, this increases the challenges of data integration, adds complexity to medical operations, and leads to issues such as complicated device management and data security vulnerabilities.

Therefore, it is hoped that an interconnection platform for internal communications among multiple applications of a physiological monitor can be developed to address the afore-mentioned challenges of data integration, complex medical operations, complicated equipment management, and data security vulnerabilities.

### SUMMARY OF THE INVENTION

In view of the issues mentioned above, the present invention provides an interconnection platform for internal communications among multiple applications of a physiological monitor. This mitigates the disadvantages and challenges associated with data integration, complex medical and nursing operations, complicated equipment management, and data security vulnerabilities.

The interconnection platform for internal communications among multiple applications of a physiological monitor comprises a physiological information application device. The physiological information application device comprises a storage unit and a processing unit. The storage unit stores at least one server application and at least one client application. The processing unit is connected to the storage unit to access the server application and the client application.

When the processing unit executes the server application, the server application establishes a connection service category, initializes a connection service, starts the connection service, and executes the connection service. When the processing unit executes the client application, the client application establishes a binding service category, initializes a binding service, executes the binding service, binds to the server application, and connects to the bound server application. When the client application connects to the bound server application, the server application interconnects with the client application through the connection service to exchange data.

The processing unit of the physiological information application device within the interconnection platform for internal communications among multiple applications of a physiological monitor executes the connection service through the server application and performs the binding service via the client application. This enables the server application and client application to interconnect and to exchange data. Consequently, data need not be transferred to external information devices, as data exchange occurs solely within the physiological information application device, thereby enhancing data security. Furthermore, since data processing occurs within the same physiological information application device, equipment management is simplified.

In order to make the above objects, features and advantages of the present invention more apparent and easier to understand, the following embodiments, together with the accompanying drawings, are described in detail as follows.

### BRIEF DESCRIPTION OF THE DRAWINGS

FIG. 1 illustrates a block diagram of an interconnection platform for internal communications among multiple applications of a physiological monitor of the present invention;

FIG. 2 illustrates a flowchart of a physiological information application device of the interconnection platform for internal communications among multiple applications of a physiological monitor of the present invention;

FIG. 3 illustrates a flowchart of a server application of the physiological information application device of the interconnection platform for internal communications among multiple applications of a physiological monitor of the present invention;

FIG. 4 illustrates a flowchart of a client application of the physiological information application device of the interconnection platform for internal communications among multiple applications of a physiological monitor of the present invention.

### DETAILED DESCRIPTION OF THE INVENTION

The technical contents, features and effects of the present invention will be clearly presented in the following detailed description of the preferred embodiment with reference to the drawings. In addition, the directional terms mentioned in the following embodiments, such as: up, down, left, right, front, back, bottom, top, etc., are only relative directions with reference to the drawings, and do not represent absolute directional positions; therefore, the directional terms used are for the convenience of illustrating their relative positional relationships, and are not intended to impose limitations on the present invention.

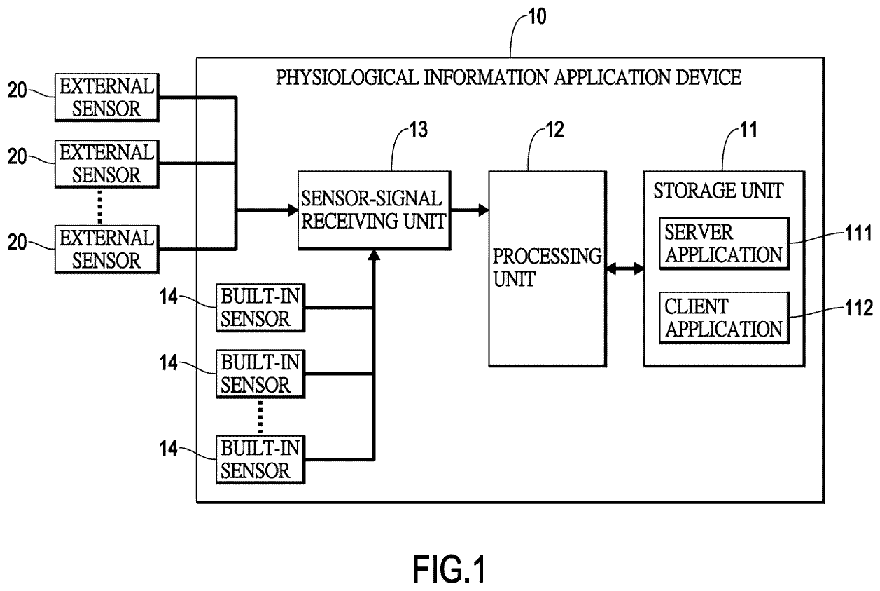

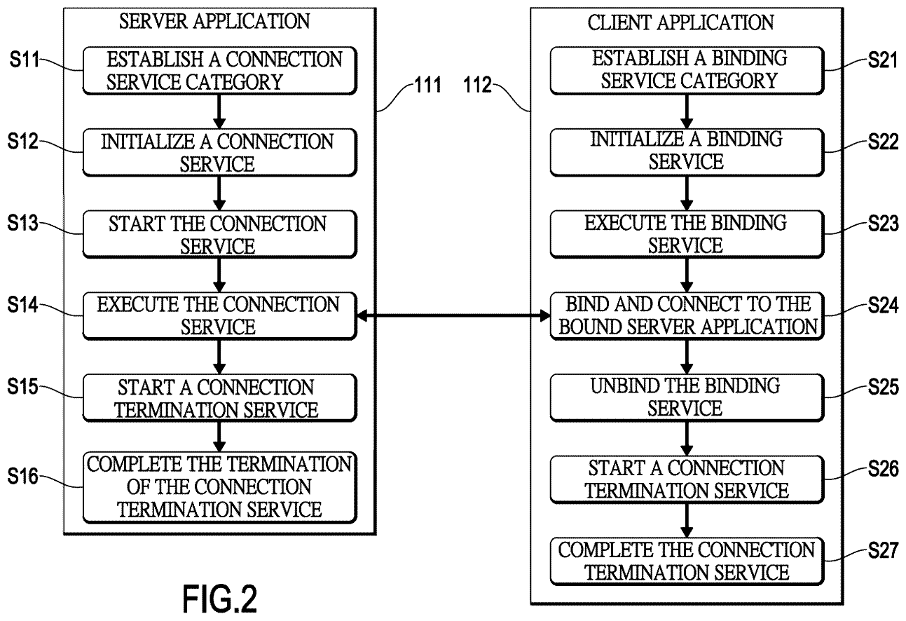

Please refer to FIGS. 1 and 2. The present invention provides an interconnection platform for internal communications among multiple applications of a physiological monitor. The interconnection platform for internal communications among multiple applications of a physiological monitor comprises a physiological information application device 10. This physiological information application device 10 comprises a storage unit 11 and a processing unit 12.

The storage unit 11 has at least one server application 111 and at least one client application 112. The processing unit 12 connects to the storage unit 11 to access the server application 111 and the client application 112.

When the processing unit 12 executes the server application 111, the server application 111 establishes a connection service category (S11), initializes a connection service (S12), starts the connection service (S13), and executes the connection service (S14). Furthermore, when the server application 111 wants to terminate the connection service, the server application 111 starts a connection termination service (S15) and completes the termination of the connection termination service (S16).

When the processing unit 12 executes the client application 112, the client application 112 establishes a binding service category (S21), initializes a binding service (S22), executes the binding service (S23), and binds and connects to the bound server application 111 (S24). Furthermore, when the client application 112 wants to terminate the binding service, the client application 112 unbinds the binding service (S25), and then the client application 112 starts a connection termination service (S26) and completes the connection termination service (S27).

When the client application 112 connects to the bound server application 111, the bound server application 111 interconnects with the client application 112 through the connection service to exchange data.

The processing unit 12 performs the connection service through the server application 111 and the binding service through the client application 112, enabling the server application 111 and the client application 112 to interconnect and exchange data. In this way, data does not need to be transferred to external information devices; data exchange can occur solely within the physiological information application device 10, thus improving data security. Furthermore, since data is processed within the same physiological information application device 10, device management is simplified.

In this embodiment, the server application 111 establishes a connection service interface comprising data formats and transmission content, as well as names, methods, input parameters and return value types of application programming interfaces (APIs). The server application 111 has two main interfaces: IBS Service (Interface BROADSIMS® Service, abbreviated as IBSService) and IBS Listener (Interface BROADSIMS® Listener, abbreviated as IBSListener). The IBSService is for communication from the server application 111 to the client application. The server application 111 can be further edited and designed to enhance flexibility. The IBSListener is for communication from the client application 112 to the server application 111. The client application 112 can also be edited and designed to enhance flexibility.

Specifically, in step S11, establishing the connection service category (start Service) involves establishing a subcategory of the connection service to override the asynchronous callback method, which is an important procedure for managing the connection service's lifecycle. Step S12 involves initializing the connection service (onCreate), which means initializing components and parameters. In step S13, the connection service is started (onStartCommand). Step S14 involves executing the connection service (Service Running), at which point the IBSService and IBSListener are in an available state. Step S15 involves initiating the connection service termination (On Destroy), which involves handling the process before the connection service is terminated. Step S16 involves completing the termination of the connection service (Service Shutdown), which means to complete the termination of the connection service.

Additionally, in step S21, establishing the binding service category means to establish a subcategory of the binding service that overrides the asynchronous callback method, and connects to the connection service of the server application 111. This interface enables components to interact with the connection service, to send requests, and to receive results. In step S22, initializing the binding service (onCreate) means the binding service's subcategory establishes initialization components and parameters. In step S23, the bound service (onBind) is executed to bind the client application 112 to the server application 111. Step S24 involves connecting to the bound server application 111 (Bound to Service), which means that the connection service is now bound and that data exchange and communication with the server application 111 can be performed through the IBSService and IBSListener categories. Step S25 involves unbinding the service (onUnBind), which unbinds the connection service. Step S26 involves starting the close connection service (on Destroy), which is a process that occurs before the connection service closes. Step S27 involves completing the service termination process (Service Shutdown), which indicates that the service termination process is complete.

Furthermore, when the server application 111 exchanges data with the client application 112 through the connection service, the multiple content data transmitted by the server application 111 to the client application 112 each have a respective serial number, and the serial numbers of these content data are all different from each other.

Furthermore, the server application 111 possesses a configuration serial number. Based on the configuration serial number, the server application 111 assigns the serial numbers to the content data when transmitting said content data to the client application 112. The server application 111 has a daily reset time, and at this daily reset time, the server application 111 resets the configuration serial number. This ensures data consistency.

For example, the storage unit 11 of the physiological information application device 10 contains a space for storing the serial number of each transmitted content data. The configuration serial number of the server application 111 is incremented by 1 each time the content data is transmitted. Consequently, these serial numbers of the content data can be used to verify whether the data streams are continuous or if there are any omissions.

In addition, the serial number also has a reset-to-zero mechanism to prevent the serial number from increasing indefinitely and to avoid reaching or exceeding an upper limit of the data format. For example, the mechanism resets the configuration serial number to zero at 00:00:00 every day according to the system time.

Furthermore, the server application 111 possesses content data to be transmitted and a content data length corresponding to the content data. The content data comprises multiple category data, and each of the multiple category data respectively has a category data length. When the server application 111 exchanges data with the client application 112 via the connection service, the server application 111 sums up the category data lengths of the multiple category data in the content data and determines whether a sum of the category data lengths matches the content data length. When the sum of the category data lengths matches the length of the content data, the server application 111 determines that the content data is correct and transmits the content data to the client application 112.

For example, each category data has its own category data length. The category data length can be used to quickly verify whether the length of the category data is incorrect, and to quickly determine the size of the category data. For instance, an electrocardiogram (ECG) category data comprises an ECG status, a heart rate value, and an ECG waveform, etc. Summing the lengths of all these data elements—the ECG status, the heart rate value, and the ECG waveform, etc. yields the ECG category data length. Furthermore, the correctness of the category data can be determined by checking the category data length.

Furthermore, when the server application 111 exchanges data with the client application 112 via the connection service, the server application 111 calculates a first hash value based on a content data to be transmitted, and sends both the content data and the first hash value to the client application 112. Upon receiving the content data and the first hash value transmitted by the server application 111, the client application 112 generates a second hash value based on the received content data and determines whether the first hash value matches the second hash value. When the first hash value matches the second hash value, the client application 112 determines that the content data is correct.

In this embodiment, both the first hash value and the second hash value are generated using an MD5 Message-Digest Algorithm.

For example, to ensure that the content data is error-free, unaltered, and complete, the present invention employs the MD5 Message-Digest Algorithm. This algorithm features the ability to process input of variable length while producing a fixed-length output of 128 bits. Furthermore, the MD5 Message-Digest Algorithm possesses the following properties:
1. Compressibility: Regardless of the length of the data, the calculated MD5 hash value always has a fixed length.
2. Ease of Computation: Calculating the MD5 hash value from the original data is straightforward.
3. Tamper Resistance: Any modification to the original data, even altering a single byte, will result in a significantly different MD5 hash value.
4. Strong Collision Resistance: It is extremely difficult to find two different data that share the same MD5 hash value.

For example: Once the server application 111 has calculated the MD5 hash value for the content data, the server application 111 transmits both the content data and the hash value together. Upon receiving the content data and the hash value, the client application 112 calculates its own MD5 hash value based on the received content data. The client application 112 then compares its own MD5 hash value with the received hash value to determine whether they match. If they match, the content data is deemed valid and usable.

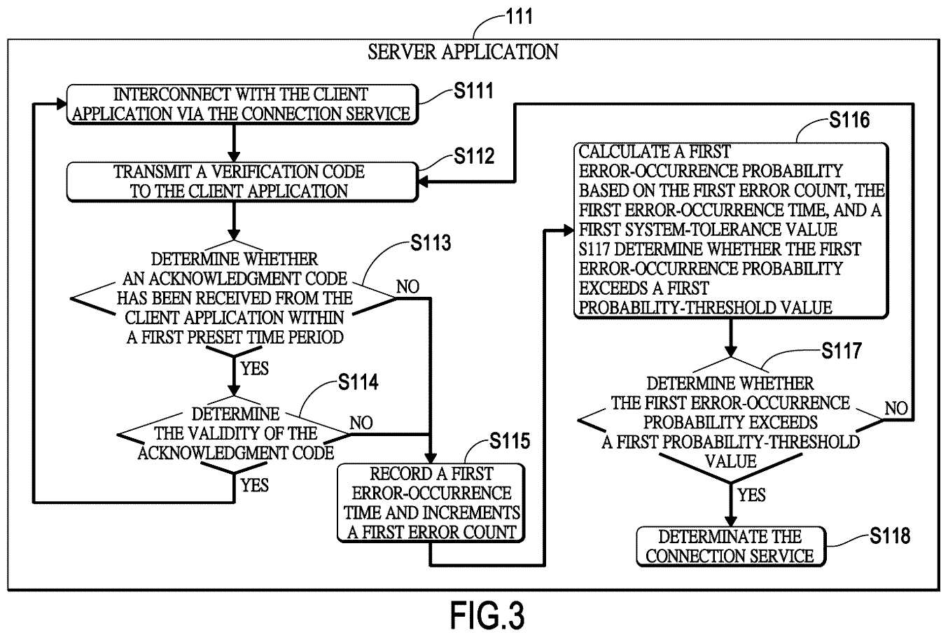

Referring to FIG. 3, when the server application 111 interconnects with the client application 112 via the connection service (S111), the server application 111 further transmits a verification code to the client application 112 (S112) and determines whether an acknowledgment code has been received from the client application 112 within a first preset time period (S113). Upon receiving the acknowledgment code within the first preset time period, the server application 111 further determines the validity of the acknowledgment code (S114). If the acknowledgment code is invalid, the server application 111 records a first error-occurrence time and increments a first error count (S115). When the server application 111 does not receive the acknowledgment code within the first preset time period, the server application 111 records the first error-occurrence time and increments the first error count (S115). After recording the first error-occurrence time and incrementing the first error count, the server application 111 further calculates a first error-occurrence probability based on the first error count, the first error-occurrence time, and a first system-tolerance value (S116), and determines whether the first error-occurrence probability exceeds a first probability-threshold value (S117). When the first error-occurrence probability exceeds the first probability-threshold value, the server application 111 terminates the connection service (S118).

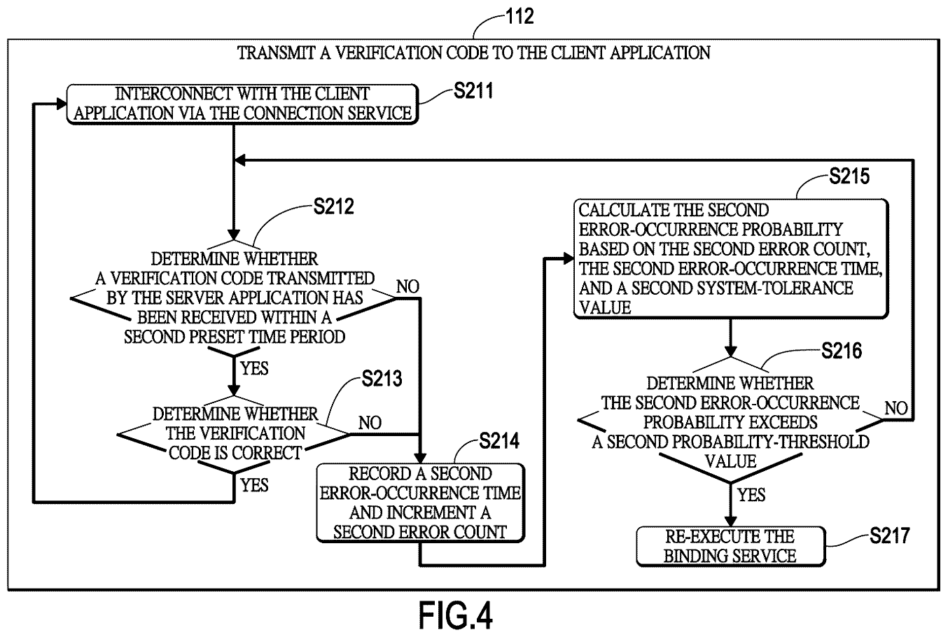

Referring to FIG. 4, when the server application 111 interconnects with the client application 112 via the connection service (S211), the client application 112 determines whether it has received a verification code transmitted by the server application 111 within a second preset time period (S212). If the client application 112 receives the verification code within the preset time, the client application 112 further determines whether the verification code is correct (S213). If the verification code is incorrect, the client application 112 records a second error-occurrence time and increments a second error count (S214). If the client application 112 fails to receive the verification code within the second preset time period, the client application 112 records the second error-occurrence time and increments the second error count (S214). After recording the second error-occurrence time and incrementing the second error count, the client application 112 further calculates the second error-occurrence probability based on the second error count, the second error-occurrence time, and a second system-tolerance value (S215), and determines whether the second error-occurrence probability exceeds a second probability-threshold value (S216). When the second error-occurrence probability exceeds the second probability-threshold, the client application 112 terminates the binding service and re-executes the binding service (S217).

For example, the server application 111 periodically transmits a verification code, such as 0x05, to the subsequent client application 112, and receives an acknowledgment code, such as 0x55 in an acknowledgment message (Ack) returned by the client application 112. When the server application 111 determines the acknowledgment code is valid i.e. 0x55 and is also returned within the first preset time period, then the server application 111 deems the acknowledgment code valid. Otherwise, the server application 111 deems the acknowledgment code invalid. After accumulating data over time, a probability of N error-occurrences can be estimated. If the probability exceeds a certain threshold, the server application 111 determines the system is unstable and initiates subsequent actions. For example, the server application 111 displays an error message and terminates the connection service. Alternatively, the client application 112 displays an error message, terminates the client application 112, restarts the client application 112, and rebinds the client application 112 to the server application 111. The present invention employs the above mechanism to prevent system-wide crashes.

Specifically, the stability assessment of the present invention adopts Poisson process analysis to perform reliability analysis based on the number of event occurrences, and thereby provides a probability of an event occurrence. The Poisson process analysis possesses the following features:
1. The occurrence of events in different time periods or specific regions is independent from each other, that is, the Poisson process is memoryless.
2. Regardless of the starting point of time or region, the probability of an event occurrence in a certain time period or a specific region is under consideration.
3. In a very short time period or a very small area, the occurrence of more than one event is extremely rare, i.e. when time or area is subdivided into extremely small units, the event will either occur only once or not at all therein.

The Poisson probability distribution function is as follows:

$$p ( x ; λ \cdot t ) = P r [ X = x ] = \frac{{( λ t )}^{x} {e}^{- λ t}}{X !} ; x = 0 , 1 , 2 , …$$

p is the probability distribution function, x is the number of occurrences, λ is the average rate at which events occur, and t is the time or space interval.

Adopting the above features, the present invention can set a threshold for the probability of N errors occurrence to infer the well-being of the system. When a probability exceeds the set threshold, the server application 111 will terminate the connection service, or the client application 112 will restart itself and reconnect and rebind to the server application 111.

In a system executing multiple applications, the connection service of the physiological information application device 10 provides a mechanism for data exchange within the system and monitors communication status without requiring data to be relayed externally, thereby reducing risks of data leakage and enhancing data confidentiality. In other words, the present invention facilitates data exchange between applications within the system. When multiple applications are interconnected via the connection service for data exchange, the connection service enables monitoring of communication status among the multiple applications after completing authentication mechanisms. The present invention also uses data stream serial numbers to determine data omissions, and uses category data lengths and hash values to determine data consistency. Once data omissions and data consistency are determined to be correct, subsequent data exchange, sharing, and application can proceed.

For example, the physiological information application device 10 is a medical device that supports the connection service and only has the server application 111 pre-installed. The server application 111 collects the physiological data. Therefore, the medical device only possesses the pre-installed functionality to collect the physiological data. The client application 112 has the connection service and a hospital system information transmission protocol function. Once the client application 112 is installed on the medical device, the medical device gains the capability to transmit data to the hospital system.

For example, as shown in FIG. 1, the physiological information application device 10 further comprises a sensor-signal receiving unit 13 and a plurality of built-in sensors 14. The sensor-signal receiving unit 13 is connected to the processing unit 12. The built-in sensors 14 are connected to the sensor-signal receiving unit 13. The processing unit 12 receives multiple physiological data generated by the built-in sensors 14 through the sensor-signal receiving unit 13. When the processing unit 12 executes the server application 111, the server application 111 further collects the physiological data. When the client application 112 connects to the bound server application 111, the client application 112 exchanges data with the server application 111 to receive the physiological data collected by the server application 111. Based on this physiological data, the client application 112 generates an application information.

Furthermore, the sensor-signal receiving unit 13 is also configured for communication with a plurality of external sensors 20. The processing unit 12 receives a plurality of physiological data generated by the external sensors 20 through the sensor-signal receiving unit 13. When the processing unit 12 executes the server application 111, the server application 111 further collects the physiological data. When the client application 112 connects to the bound server application 111, the client application 112 exchanges data with the server application 111 to receive the physiological data collected by the server application 111. Based on the physiological data, the client application 112 generates application information.

In summary, the present invention can monitor the status of communication among multiple applications in real time and take immediate action when an anomaly is detected, thus preventing system crashes and increasing system stability. Furthermore, the present invention can ensure the consistency of exchanged data, preventing misuse of incorrect data. When subsequent functional expansion or addition of extended applications is required, only the corresponding client application 112 needs to be installed, thus eliminating the need for additional information equipment and data transfer to external systems. Consequently, data confidentiality is enhanced, and data reuse becomes more advantageous.

The present invention utilizes data stream serial numbers to ensure data completeness and employs data length and hash values to guarantee data integrity. To ensure system stability, the present invention further employs a Poisson probability distribution function to monitor communication status among applications and estimates the probability of an Nth error occurring in the system, which can serve as a basis for system stability. When the estimated number of errors and its probability exceed the system's tolerance threshold, appropriate measures are taken to prevent system failure.

Although the present invention has been disclosed as above by way of a preferred embodiment, it is not intended to limit the present invention, and any one skilled in the art may make certain changes and modifications without departing from the spirit and scope of the present invention, and therefore the scope of protection of the present invention shall be subject to the scope of the appended patent claims as defined herein.

What is claimed is:

1. An interconnection platform for internal communications among multiple applications of a physiological monitor, the interconnection platform comprising:
a physiological information application device, comprising:
a storage unit storing at least one server application and at least one client application;
a processing unit connected to the storage unit to access the server application and the client application;
wherein, when the processing unit executes the server application, the server application establishes a connection service category, initializes a connection service, starts the connection service, and executes the connection service;
wherein, when the processing unit executes the client application, the client application establishes a binding service category, initializes a binding service, executes the binding service, binds to the server application, and connects to the bound server application;
wherein, when the client application connects to the bound server application, the server application interconnects with the client application through the connection service for data exchange.

2. The interconnection platform for internal communications among multiple applications of a physiological monitor as claimed in claim 1, wherein when the server application exchanges data with the client application through the connection service, multiple content data transmitted by the server application to the client application are each assigned a respective serial number, and these serial numbers of the content data are distinct from each other.

3. The interconnection platform for internal communications among multiple applications of a physiological monitor as claimed in claim 2, wherein the server application has a configuration serial number and sets the serial numbers for the content data when transmitting the content data to the client application based on the configuration serial number;
wherein the server application increments the configuration serial number after each transmission of the content data;
wherein the server application has a daily reset time, and the server application resets the configuration serial number at the daily reset time.

4. The interconnection platform for internal communications among multiple applications of a physiological monitor as claimed in claim 1, wherein the server application has a content data to be transmitted and a content data length corresponding to the content data, the content data comprises multiple category data, each with its own category data length;
when the server application exchanges data with the client application via the connection service, the server application sums up the category data lengths of the multiple category data within the content data and determines whether a sum of the category data lengths of the multiple category data matches the content data length;
wherein, when the sum of the category data lengths of the multiple category data matches the content data length, the server application determines that the content data is valid and transmits the content data to the client application.

5. The interconnection platform for internal communications among multiple applications of a physiological monitor as claimed in claim 1, wherein
when the server application exchanges data with the client application through the connection service, the server application calculates a first hash value of a content data to be transmitted and sends both the content data and the first hash value to the client application;
upon receiving the content data and the first hash value transmitted by the server application, the client application generates a second hash value based on the content data and determines whether the first hash value matches the second hash value;
when the first hash value matches the second hash value, the client application determines that the content data is correct.

6. The interconnection platform for internal communications among multiple applications of a physiological monitor as claimed in claim 5, wherein both the first hash value and the second hash value are generated using an MD5 message-digest algorithm.

7. The interconnection platform for internal communications among multiple applications of a physiological monitor as claimed in claim 1, wherein
when the server application interconnects with the client application via the connection service, the server application further transmits a verification code to the client application and determines whether an acknowledgment code returned by the client application is received within a first preset time period;
when the server application receives the acknowledgment code within the first preset time period, the server application further determines whether the acknowledgment code is correct;
wherein, when the acknowledgment code is incorrect, the server application records a first error-occurrence time and increments a first error count;
wherein, when the server application fails to receive the acknowledgment code within the first preset time period, the server application records the first error-occurrence time and increments the first error count;
wherein, after recording the first error-occurrence time and incrementing the first error count, the server application further calculates a first error-occurrence probability based on the first error count, the first error-occurrence time, and a first system-tolerance value, and determines whether the first error-occurrence probability exceeds a first probability-threshold value;
wherein, when the first error-occurrence probability exceeds the first probability-threshold value, the server application terminates the connection service.

8. The interconnection platform for internal communications among multiple applications of a physiological monitor as claimed in claim 1, wherein
when the server application interconnects with the client application through the connection service, the client application determines whether the client application has received a verification code transmitted by the server application within a second preset time period;
wherein, when the client application receives the verification code within the second preset time period, the client application further determines whether the verification code is correct;
wherein, when the verification code is incorrect, the client application records a second error-occurrence time and increments a second error count;
wherein, when the client application fails to receive the verification code within the second preset time period, the client application records the second error-occurrence time and increments the second error count;
after recording the second error-occurrence time and incrementing the second error count, the client application further calculates a second error-occurrence probability based on the second error count, the second error-occurrence time, and a second system-tolerance value, and determines whether the second error-occurrence probability exceeds a second probability-threshold value;
wherein, when the second error-occurrence probability exceeds the second probability-threshold value, the client application terminates the binding service and re-executes the binding service.

9. The interconnection platform for internal communications among multiple applications of a physiological monitor as claimed in claim 1, wherein the physiological information application device further comprises:
a sensor-signal receiving unit connected to the processing unit;
a plurality of built-in sensors connected to the sensor-signal receiving unit;
wherein the processing unit receives a plurality of physiological data generated by the built-in sensors through the sensor-signal receiving unit;
wherein, when the processing unit executes the server application, the server application further collects the physiological data;
wherein, when the client application connects to the bound server application, the client application exchanges data with the server application to receive the physiological data collected by the server application, and the client application generates an application information based on the physiological data.

10. The interconnection platform for internal communications among multiple applications of a physiological monitor as claimed in claim 1, wherein the physiological information application device further comprises:
a sensor-signal receiving unit connected to the processing unit and configured for communication with a plurality of external sensors;
wherein the processing unit receives a plurality of physiological data generated by the external sensors through the sensor-signal receiving unit;
wherein, when the processing unit executes the server application, the server application further collects the physiological data;
wherein, when the client application connects to the bound server application, the client application exchanges data with the server application to receive the physiological data collected by the server application, and the client application generates an application information based on the physiological data.
